(19) United States
(12) Patent Application Publication
(10) Pub. No.: US 2021/0369299 A1
(43) Pub. Date: Dec. 2, 2021

(54) DEVICE AND METHOD FOR PROVIDING ANTEGRADE VASCULAR ACCESS THROUGH RETROGRADE APPROACH

(71) Applicant: Murugan Athigaman, Carlsbad, NM (US)

(72) Inventor: Murugan Athigaman, Carlsbad, NM (US)

(21) Appl. No.: 17/302,471

(22) Filed: May 4, 2021

Related U.S. Application Data
(60) Provisional application No. 62/985,040, filed on Mar. 4, 2020.

(51) Int. Cl. A61B 17/34

(52) U.S. Cl. CPC A61B 17/3415; A61B 2017/00292

(57) ABSTRACT

A device for providing antegrade vascular access through retrograde approach is disclosed. The device includes a balloon arranged on a sheath and one or more side holes formed in the sheath proximal of the balloon. The balloon may be bulbous. A method for providing antegrade vascular access through retrograde approach is also disclosed.

### CROSS-REFERENCE TO RELATED APPLICATION

This application claims the benefit of priority of U.S. provisional application number 62/985,040, filed Mar. 4, 2020, the contents of which are herein incorporated by reference.

### BACKGROUND OF THE INVENTION

The present invention is related to endovascular interventions and, more particularly, to devices and methods that can be used to provide antegrade access in various endovascular interventions.

Catheter angiography is a form of treatment utilized with patients exhibiting Peripheral Artery Disease (“PAD”). The most common mode of access for these types of peripheral interventions is through a retrograde manner, such as in the common femoral artery, but there are certain limitations to this access. For example, distal lesions below the knee could be difficult to reach, and also heavily calcified and tortuous iliac vessels, previous abdominal aortic graft with a steep angle, and previous endoluminal graft in the Aorta may inhibit access. Because retrograde access is difficult in these circumstances, the physician may instead utilize an antegrade approach.

However, current devices and methods utilized for providing antegrade access to the femoral artery have substantial risk of bleeding locally and into the abdomen and retroperitoneum, and such complications may be lethal. In addition, endovascular procedures involving patients with arterio-venous (“AV”) grafts (e.g., dialysis patients) generally involve two different access sites with twice the risk of access site complications.

Recent techniques for antegrade access through retrograde approach (including those described by August Ysa, MD, et al. in Vascular and Endovascular Techniques, “A new simple method of converting a retrograde femoral access to an antegrade catheterization”), however, are cumbersome.
Thus, a device and method for providing antegrade vascular access through a retrograde approach may be beneficial.

### SUMMARY OF THE INVENTION

In one aspect of the present invention, an endovascular device includes: a sheath that defines a lumen extending between a distal opening and a proximal opening; a balloon arranged on the sheath; a balloon port in communication with the balloon for dilating the balloon; and at least one side hole formed in the sheath proximal to the balloon for receiving a guide wire.

In another aspect of the present invention, an endovascular system includes: a guide wire configured to be inserted into a blood vessel in a first direction; and an endovascular device including: a sheath that defines a lumen extending between a distal opening and a proximal opening, the guide wire extending at least partially through the sheath; a balloon arranged on the sheath; a balloon port in communication with the balloon for dilating the balloon; and at least one side hole formed in the sheath proximal to the balloon for receiving the guide wire and for advancing the guide wire in a second direction when the balloon is dilated.

In yet another aspect of the present invention, a method for providing antegrade vascular access through a retrograde approach includes the steps of: introducing a first sheath to a blood vessel through a puncture site; inserting a guide wire into the blood vessel in a first direction; exchanging the first sheath for an endovascular device including: a second sheath that defines a lumen extending between a distal opening and a proximal opening; a balloon arranged on the second sheath; a balloon port in communication with the balloon for dilating the balloon; and at least one side hole formed in the second sheath proximal to the balloon; withdrawing the guide wire; dilating the balloon; reintroducing the guide wire, the guide wire being received by one of the at least one side holes and advanced in a second direction.

These and other features, aspects and advantages of the present invention will become better understood with reference to the following drawings, description and claims.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 illustrates an exemplary endovascular device in accordance with the present invention;

FIG. 2 illustrates a partial view of the device of FIG. 1 having an alternate balloon configuration;

FIG. 3A illustrates a first step of an exemplary method of using the endovascular device to provide antegrade access;

FIG. 3B illustrates a second step of the exemplary method of using the endovascular device to provide antegrade access, with portions of the endovascular device not shown, for clarity;

FIG. 3C illustrates a third step of the exemplary method of using the endovascular device to provide antegrade access, with portions of the endovascular device not shown, for clarity;

FIG. 3D illustrates a fourth step of the exemplary method of using the endovascular device to provide antegrade access, with portions of the endovascular device not shown, for clarity;

FIG. 4A illustrates a first step of an exemplary method for accessing arteriovenous (AV) grafts through a single access site;

FIG. 4B illustrates a second step of the exemplary method for accessing arteriovenous (AV) grafts through the single access site; and

FIG. 4C illustrates a third step of the exemplary method for accessing arteriovenous (AV) grafts through a single access site.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The embodiments described herein provide an endovascular device and method that provides antegrade vascular access through a retrograde approach. These devices and methods may be utilized in various endovascular scenarios, including but not limited to PAD treatments, percutaneous interventions, and various other endovascular interventions, including but not limited to those where multiple balloons and/or special balloon geometry may be beneficial.

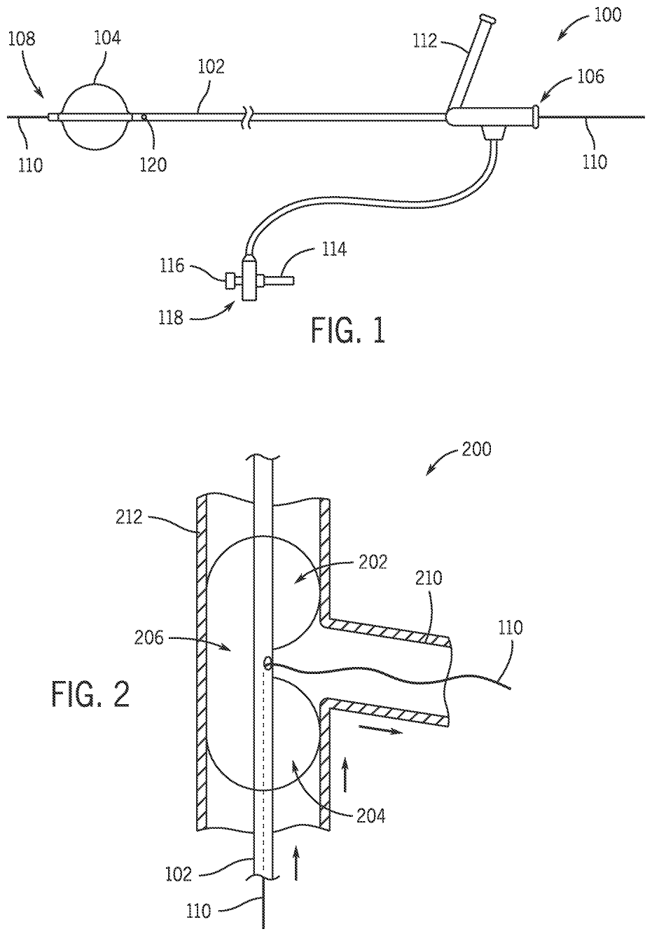

FIG. 1 illustrates an endovascular device 100 (hereinafter, “the device 100”) according to one or more embodiments of the present disclosure. The device 100 facilitates access antegrade and retrograde and thus mitigates potential risks associated with conventional devices and methods.

In the illustrated example, the device 100 includes a sheath 102, a soft balloon 104 incorporated in the sheath 102, a lumen (obscured from view, but understood to be housed within the sheath 102) extending through the sheath 102 with a proximal opening 106 and a distal opening 108 for guide wire 110 access, a side port 112 (also referred to as a balloon port) for dilation of the balloon 104, a side port 114 for flushing and aspiration of the proximal opening 106 and having a locking mechanism 116 to prevent any bleeding or air embolism, and a 3-way stop cock 118 to provide 3-way flow. The lumen is embodied as a soft segment, and its operation described in greater detail below. In addition, the sheath 102 includes one or more side holes 120 proximal to the balloon 104. In use, once balloon 104 is inflated/dilated, the guide wire 110 will U-turn and head out of one of the side holes 120. The side port 112 only communicates with the balloon 104. The side port 114 only communicates with proximal 106 and distal openings 108 through the balloon 104, for example, when aspirating blood to keep the sheath 102 clean.

In some examples, the balloon 104 is a soft balloon, for example made of latex or other soft materials; however, hard material balloons may be utilized as well, in addition to or instead of a soft balloon. The purpose of the balloon 104 is to close the arterial lumen to redirect the guide wire 110 through the side hole 120. In some examples, two or more balloons 104 may be utilized, for example, proximal and distal to the side holes 120. In some examples, the balloon 104 may be bulbous, such that the balloon 104 may engage a wall of an artery (or vein) at more than one location. For example, the balloon 104 may have two or more bulbous portions having greater cross-section than the non-bulbous portions. This may be advantageous in providing access to side branches in arteries or veins.

FIG. 2 illustrates a bulbous balloon 200 according to one or more embodiments. Here, the bulbous balloon 200 is configured with a “bean” shaped geometry and includes a pair of bulbous portions 202, 204 having greater cross-section than a non-bulbous portion 206. With this configuration, the bulbous balloon 200 provides access to a side branch 210 of a vein 212 or artery. In some examples, the bulbous portion 202 may be inflated first, and then the guide wire 110 is directed into the non-bulbous portion 206, and then the other bulbous portion 204 is inflated, and then the guide wire 110 may be advanced through the side holes 110. Here, markers could be included on the non-bulbous portion 206 and/or the side holes, or the non-bulbous portion 206 and/or the side holes may be opaque to permit visualization through X-ray or fluoroscopy.

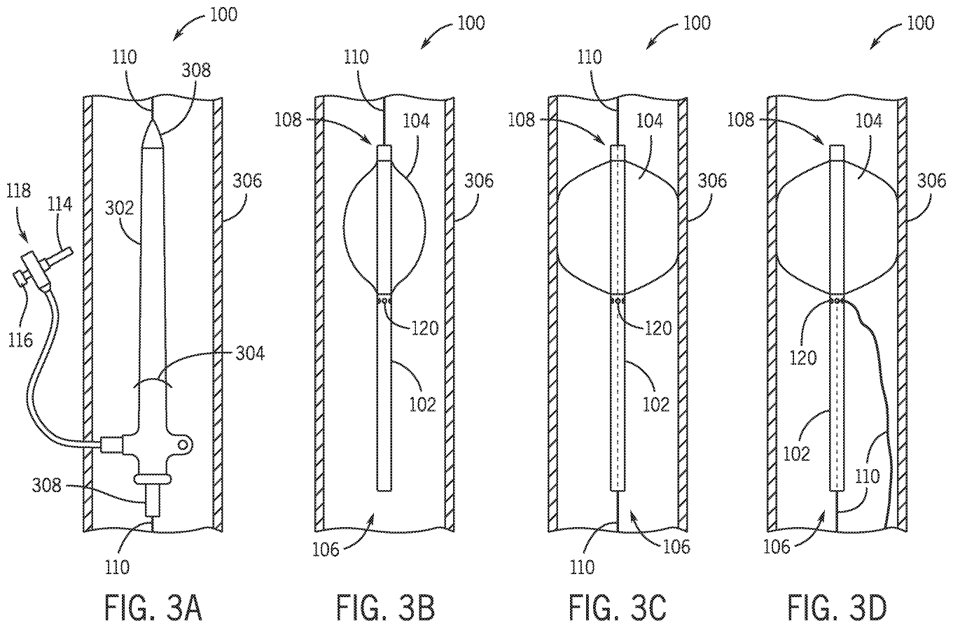

Also disclosed herein are antegrade access methods. Such methods may utilize the device 100, described above, in combination with other endovascular devices. FIGS. 3A-3D illustrates an antegrade access method according to one or more embodiments of the present disclosure. For example, a conventional sheath 302 with a dilator 308 at end thereof is used first. The conventional sheath 302 is introduced to a blood vessel 306 (e.g., a common femoral artery) through a puncture site 304 (e.g., a femoral artery puncture site) via the dilator 308. After accessing the blood vessel 306 using the conventional sheath 302 (FIG. 3A), the guide wire 110 is left in place. The standard sheath 302 is then exchanged for the sheath 102 of the device 100 (FIG. 3B) via the puncture site 304 (although the puncture site 304 is not shown in FIGS. 3B-3D, sheath 102 is also inserted through it). The position of the sheath 102 may then be checked with a quick contrast injection. The balloon 104 is then inflated (FIG. 3C). The guide wire 110 is withdrawn and, as the inflated balloon 104 collapses the lumen (the soft segment) of the sheath 102, the guide wire 110 is reintroduced (FIG. 3D). Consequently, the guide wire 110 is then directed towards and out one of the proximal side holes 120 in the sheath 102 (FIG. 3D). This guide wire 110 is subsequently advanced antegrade into the distal blood vessel 306. Once this is achieved and confirmed by further contrast injection, the device 100 is removed and exchanged for a standard vascular sheath 302 being directed antegrade. Now that the access is achieved antegrade, the planned endovascular procedure is completed. Subsequently the process could be repeated, and the wire 110 advanced proximally and used for any standard closure device. In addition, a buddy wire technique could be utilized to provide access both antegrade and retrograde.

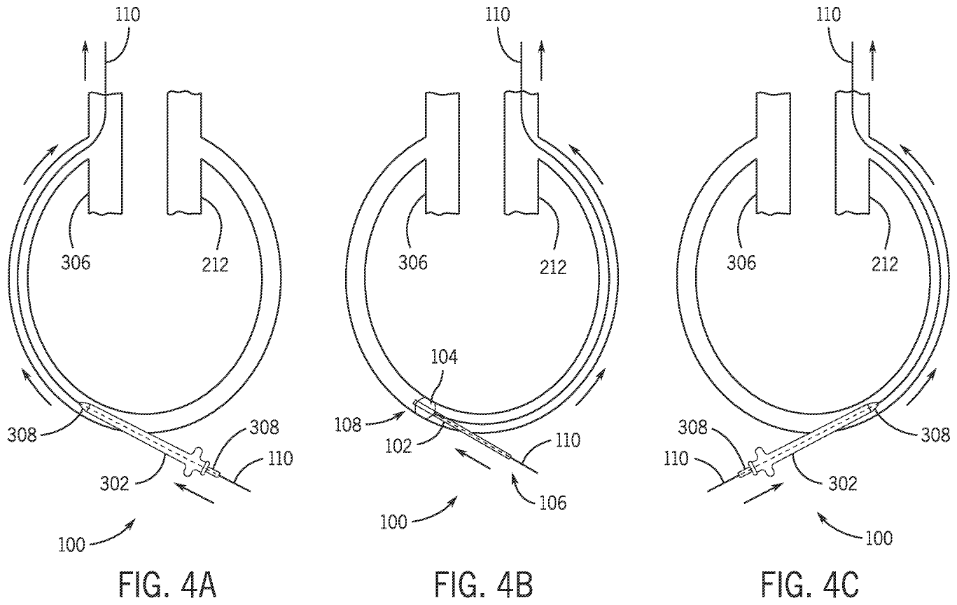

This technique could potentially be used in other areas like dialysis access AV grafts where instead of making two different access sites, one access site could be used for both proximal and distal diagnostic study, thrombectomy, balloon dilation and stent placement. Conventional methods of accessing AV grafts during endovascular interventions require multiple access sites, whereas FIGS. 4A-4C illustrate a method for accessing AV grafts in multiple directions through a single access site, according to one or more embodiments of the present disclosure. The operation of the device 100 in FIGS. 4A-4D is equivalent to that of FIGS. 3A-3D. As shown in FIG. 4A, the conventional sheath 302 is introduced to a blood vessel and directed towards one of an arterial/venous end 306 via the dilator 308. After accessing the blood vessel 306 using the conventional sheath 302, the guide wire 110 is left in place. The standard sheath 302 is then exchanged for the sheath 102 of the device 100 (FIG. 4B). The position of the sheath 102 may then be checked with a quick contrast injection. The balloon 104 is then inflated (FIG. 4B). The guide wire 110 is withdrawn and, as the inflated balloon 104 collapses the lumen of the sheath 102, the guide wire 110 is reintroduced. The guide wire 110 is then directed towards one of the proximal side holes 120 in the sheath 102. This guide wire 110 is subsequently advanced antegrade towards the other 212 of the arterial/venous end (FIG. 4B). Once this is achieved and confirmed by further contrast injection, the device 100 is removed and exchanged for a standard vascular sheath 302 being directed antegrade (FIG. 4C).

Device and method embodiments described herein may also be used in other endovascular procedures requiring directional guide wire advancements, with the addition of more balloons.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, various appropriate methods of making may be used and would be apparent to those skilled in the art having the benefit of the teachings herein. Even furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein.

While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

The terms “proximal” and “distal” are defined herein relative to a vascular system of a patient. The term “proximal” refers to the position of an element closer to the heart of patient and the term “distal” refers to the position of an element further away from the heart of the patient. Moreover, the use of directional terms such as above, below, upper, lower, upward, downward, left, right, and the like are used in relation to the illustrative embodiments as they are depicted in the figures, the upward or upper direction being toward the top of the corresponding figure and the downward or lower direction being toward the bottom of the corresponding figure.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

What is claimed is:

1. An endovascular device comprising:
a sheath that defines a lumen extending between a distal opening and a proximal opening;
a balloon arranged on the sheath;
a balloon port in communication with the balloon for dilating the balloon; and
at least one side hole formed in the sheath proximal to the balloon for receiving a guide wire.

2. The endovascular device of claim 1, wherein the balloon is a bulbous balloon.

3. The endovascular device of claim 2, wherein the bulbous balloon includes a first and second bulbous portion each having greater cross section than a non-bulbous portion of the balloon.

4. The endovascular device of claim 1, wherein the lumen is collapsible.

5. The endovascular device of claim 1, wherein the lumen is configured to collapse when the balloon is dilated.

6. The endovascular device of claim 1, wherein the balloon is arranged proximal the distal opening.

7. An endovascular system comprising:
a guide wire configured to be inserted into a blood vessel in a first direction; and
an endovascular device comprising:
a sheath that defines a lumen extending between a distal opening and a proximal opening, the guide wire extending at least partially through the sheath;
a balloon arranged on the sheath;
a balloon port in communication with the balloon for dilating the balloon; and
at least one side hole formed in the sheath proximal to the balloon for receiving the guide wire and for advancing the guide wire in a second direction when the balloon is dilated.

8. The endovascular system of claim 7, wherein the lumen is configured to collapse when the balloon is dilated.

9. A method for providing antegrade vascular access through a retrograde approach, the method comprising the steps of:
introducing a first sheath to a blood vessel through a puncture site;
inserting a guide wire into the blood vessel in a first direction;
exchanging the first sheath for an endovascular device comprising:
a second sheath that defines a lumen extending between a distal opening and a proximal opening;
a balloon arranged on the second sheath;
a balloon port in communication with the balloon for dilating the balloon; and
at least one side hole formed in the second sheath proximal to the balloon;
withdrawing the guide wire;
dilating the balloon; and
reintroducing the guide wire, the guide wire being received by one of the at least one side holes and advanced in a second direction.

10. The method of claim 9, wherein, after the dilating the balloon step, the method further comprises the step of: collapsing the lumen.
